(19) United States
(12) Patent Application Publication
(10) Pub. No.: US 2020/0261250 A1
(43) Pub. Date: Aug. 20, 2020

(54) STENT DELIVERY SYSTEM WITH COMBINED FLUSHING PORT

(71) Applicant: Cook Medical Technologies LLC, Bloomington, IN (US)

(72) Inventor: Ryan Bradway, Tacoma, WA (US)

(21) Appl. No.: 16/792,577

(22) Filed: Feb. 17, 2020

Related U.S. Application Data
(60) Provisional application No. 62/807,032, filed on Feb. 18, 2019.

(51) Int. Cl. A61F 2/966; A61F 2/82; A61M 3/02

(52) U.S. Cl. CPC A61F 2/966; A61F 2210/0014; A61M 3/0262; A61F 2/82

(57) ABSTRACT

A stent delivery system includes a cannula with a proximal end separated from a distal carrier segment by an unbroken inner surface and an unbroken outer surface. The distal carrier segment is attached to a tip, and the proximal end of the cannula is attached to a hub. A pusher is co-axially mounted about the cannula and has a proximal end attached to the hub. A retractable sheath is co-axially mounted about the cannula and the pusher, and is movable between a first position covering the distal carrier segment and a second position uncovering the distal carrier segment. A self expanding stent is mounted about the distal carrier segment. The hub includes exactly one flushing port in fluid communication with a lumen of the cannula and annular passage between the cannula and the pusher.

### TECHNICAL FIELD

The present disclosure relates generally to stent delivery systems, and more particularly to a stent delivery system with a combined flushing port for flushing the entire device prior to use from a single flushing port.

### BACKGROUND

While current stent delivery systems have a proven track record of reliability, robustness and successful outcomes, next generation stent delivery systems often must perform equal to or better than their predecessors while potentially reducing part count, improving manufacturability and improving user experiences. In one class of stent delivery systems, a central cannula includes a lumen for receiving a guide wire to direct the device to a delivery site. The cannula also includes a distal carrier segment about which a self expanding stent is compressed under a retractable sheath.

Prior to inserting the device into a patient, it is customary to flush both the internal lumen and the co-axial passages outside of the cannula lumen, which includes the distal carrier segment and the volume therearound where the compressed stent is located. As is well known, this flushing procedure is utilized to avoid air bubbles being accidently set loose in a patient's vascular system when the device is being maneuvered to a delivery site and during stent deployment. In one class of stent delivery systems, the lumen of the cannula and the area outside of the cannula are fluidly isolated from one another, requiring that the device be flushed from two separate flushing ports. The requirement to flush the device from two separate ports can be cumbersome and inherently requires more time.

The present disclosure is directed toward one or more of the problems set forth above.

### SUMMARY

In one aspect, a stent delivery system includes a cannula having an unbroken inner surface and an unbroken outer surface that separate a proximal end from a distal carrier segment. A tip is attached to the distal carrier segment, and a hub is attached to the proximal end of the cannula. A pusher is co-axially mounted about the cannula, and has a proximal end attached to the hub. A retractable sheath is co-axially mounted about the cannula and the pusher, and is movable between a first position covering the distal carrier segment and a second position uncovering the distal carrier segment. A self expanding stent is mounted about the distal carrier segment and covered by the sheath in the first position. The stent delivery system includes exactly one flushing port that is defined by the hub and is in fluid communication with a lumen of the cannula and an annular passage between the cannula and the pusher.

In another aspect, a method of flushing a stent delivery system includes connecting a syringe to a flushing port of the stent delivery system. Flushing fluid is moved from the syringe into a first flush passage and a second flush passage defined partially by a hub that includes the flushing port. The moving step continues until the flushing fluid emerges from a distal end of the first flush passage and a distal end of the second flush passage. The flushing fluid moving step includes moving a first volume of flushing fluid along an unbroken inner surface of a cannula of the stent delivery system, and moving a second volume of flushing fluid, which is exclusive of the first volume, along an unbroken outer surface of the cannula.

### BRIEF DESCRIPTION OF THE DRAWINGS

FIG. 1 is a side elevational view of a stent delivery system according to the present disclosure;

FIG. 2 is a sectioned schematic view of the hub for the stent delivery system of FIG. 1 as viewed along sectioned lines 2-2 of FIG. 1;

FIG. 3 is a partial schematic sectioned view of the distal carrier segment portion of the stent delivery system of FIG. 1 as viewed along sectioned lines 3-3 of FIG. 1; and

FIG. 4 is an enlarged schematic sectioned view of the circled region of FIG. 3.

### DETAILED DESCRIPTION

A stent delivery system 20 is constructed about a cannula 40 that has a proximal end 43 separated from a distal carrier segment 44 by an unbroken inner surface 41 and an unbroken outer surface 42. Cannula 40 is preferably metallic, such as a nitinol tube, but could be made from other materials, including but not limited to medical grade plastics and composites. As used in this disclosure, an unbroken inner surface and an unbroken outer surface mean that there are no through holes through the wall of the cannula that defines its lumen 45 and the space around cannula 40. Thus, the cannula with the unbroken inner surface and an unbroken outer surface opens only through the proximal and distal ends of its lumen 45, and no where else. By employing such a cannula 40 in a stent delivery system 20 of the present disclosure, substantial cost savings may be achieved by avoiding any through hole machining or other processing steps other than having a nitinol tube cut to length for the specific stent delivery system 20 application. The proximal end 43 of cannula 40 is attached to a hub 30 (body piece 33, but not body piece 34) in any suitable manner, such as by adhesives and/or a friction fit in contact connection bore 35, which may be the only location that cannula 40 comes into contact with hub 30. Prior to use and when being maneuvered to a treatment location within a patient, a self expanding stent 80 is mounted about distal carrier segment 44 of cannula 40. A tip 50 is attached to and extends distally away from the distal carrier segment 44.

A pusher 60, which may take the form of a hollow tube, is co-axially mounted about cannula 40, and has a proximal end 61 attached to the hub. In particular, pusher 60 may be attached to hub 30 (body piece 34, but not body piece 33), such as by adhesives and/or a friction fit in contact connection bore 36, which is defined by hub 30. Thus, the portion of the hub 30 that defines contact connection bore 36 may be the only location of contact between pusher 60 and hub 30. Together the interior surface of pusher 60 and the external unbroken outer surface 42 of cannula 40 define an annular passage 62 that extends from hub 30 through the distal end of pusher 60, which terminates proximal to the distal carrier segment 44 of cannula 40 in a conventional manner. A flushing fluid connection 63 may extend between the annular passage 62 and the self expanding stent 80 positioned about distal carrier segment 44 of cannula 40. This may be accomplished by including one or more side ports 64 through the side wall of pusher 60.

A retractable sheath 70 is co-axially mounted about the cannula 40 and the pusher 60, and is movable between a first position 71 covering the distal carrier segment 44, and a second position 72 uncovering the distal carrier segment 44. Thus, those skilled in the art will appreciate that retractable sheath is maintained at the first position 71 prior to use and when being maneuvered to a treatment location, but is moved or slid to the second position 72 in order to deploy a self expanding stent 80 at a treatment location in a convention manner. Self expanding stent 80, which is mounted about the distal carrier segment 44 will be covered by the retractable sheath 70 when in its first position 71. Movement of the sheath 70 between the first position 71, and the second position 72 may be accomplished by a user gripping a sheath handle 75 that is located at the proximal end of the sheath 70. A distal end 73 of retractable sheath 70 may abut tip 50 so that an outer diameter of tip 50 makes a smooth transition to the outer surface of retractable sheath 70 when in its first position 71.

Hub 30 may consist of exactly two plastic bodies 33 and 34 joined end to end in a bonded connection. The stent delivery system 20 includes exactly one flushing port 31, which is defined by hub 30, and preferably located at a proximal end of the hub in body piece 33. Hub 30 may include a suitable fitting connection, such as luer lock, at the flushing port 31. Hub 30 may define one or more (two shown) wire ports 32 that may facilitate control wires associated with delivery of the self expanding stent 80. In particular, one or more wires 85 may extend between the self expanding stent 80 through the annular passage 62 and exit hub 30 at respective wire ports 32. Wire ports 32 may be sealed in a conventional manner such as with cylindrically shaped silicone seals 39 that are biased to cover and close the respective wire ports 32. The flushing port 31 is in fluid communication with lumen 45 of cannula 40, such as by opening directly into the proximal end of cannula 40 as shown. This allows a portion of the fluid pushed into hub 30 from flushing port 31 to flush through lumen 44 into lumen 45 of cannula 40 and out through a distal end 51 of tip 50. Flushing port 31 is also fluidly connected to annular passage 62, such as by a segment 13 of a bypass passageway that is located in hub 30 between contact connection bore 35 and contact connection bore 36 along centerline 46. The segment 13 maybe oriented perpendicular to centerline 46.

Stent delivery system 20 can be thought of as including a first flush passage 11 that starts at the flushing port 31, extends through the lumen 35 of cannula 40 and terminates at the distal end 51 of tip 50. A second flush passage 12 extends from the flushing port 31, along a segment of the outer surface of cannula 40 within hub 30, along the annular passage 62, between the distal carrier segment 44 and the retractable sheath 70, and terminates at a distal end 73 of retractable sheath 70. Some means is provided for channeling a substantial fraction of flushing fluid through the second fluid passage 12. This means can be accomplished structurally by avoiding flow restrictions in the second flush passage 12, or maybe by temporarily creating a blockage or flow restriction in the first flush passage 11. For instance, a removable plug 86 may be temporarily in contact with tip 50 and at least partially block the first fluid passage 11 during a flushing procedure prior to using stent delivery system 20. The plug 86 can urge some of the flushing fluid into second fluid passage 12.

### INDUSTRIAL APPLICABILITY

The stent delivery system 20 of the present disclosure finds potential application for delivering self expanding stents to passageways within a live body. The stent delivery system 20 finds particular application in the delivery of self expanding stents, and maybe stent grafts, to the vascular system of a patient.

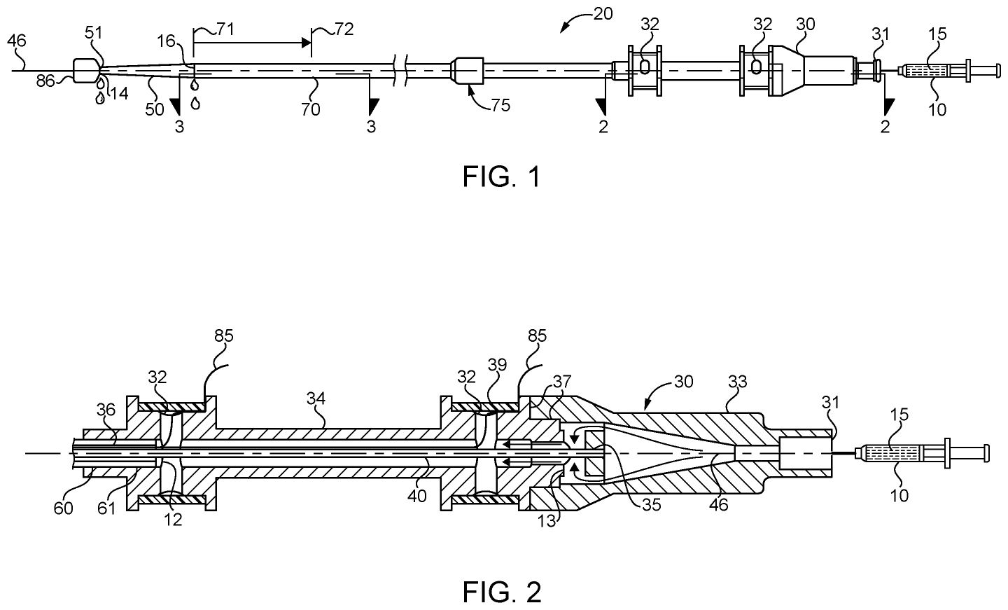

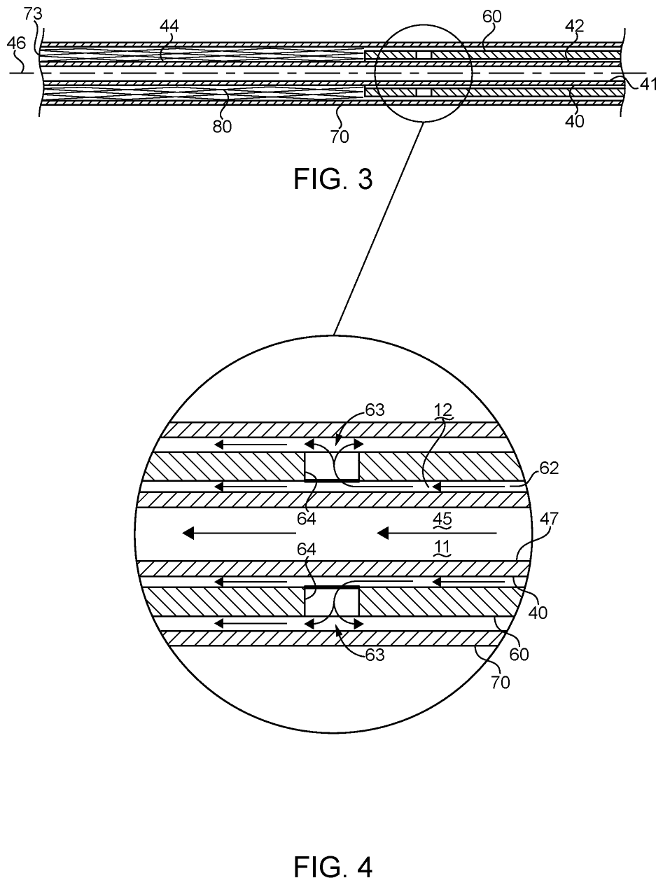

Those skilled in the art that, just prior to use, the stent delivery system 20 may be flushed with an appropriate fluid, such as saline, in order to avoid the presence of any air bubbles that could escape into a patient's circulatory system during a stent delivery procedure. A method of flushing stent delivery system 20 may begin by connecting a syringe 10 containing the flushing fluid to the flushing port 31. Flushing fluid 15 is moved from the syringe 10 into the first flush passage 11 and the second flush passage 12, which are defined at least partially by the hub 30, which includes the flushing port 31. The flushing fluid moving step is continued until flushing fluid emerges from a distal end 14 of first flush passage 11 and from a distal end 16 of the second flush passage 12 (see FIG. 1). In order for this to occur, a first volume of flushing fluid is moved along the unbroken inner surface 41, or lumen 45, of cannula 40, and a second volume of flushing fluid, which is exclusive of the first volume, is moved along the unbroken outer surface 42 of cannula 40 in the annular passage 62. Depending upon flow areas, flow restrictions and flow path lengths, it may be necessary to encourage flushing fluid into the second fluid passage 12 by at least partially blocking the first fluid passage 11. This may be accomplished by temporarily positioning a removable plug 86 in contact with tip 50 so that flushing fluid injected from syringe 10 is urged into the second fluid passage 12. When this occurs, the flushing fluid moving in the second fluid passage 12 temporarily may move perpendicular to centerline 46 of cannula 40, such as when moving into segment 13 of the second fluid passage 12 which is defined by hub 30.

The present disclosure allows for a clinician to easily observe and confirm that the stent delivery system 20 is properly flushed when flushing fluid emerges from both the distal end 14 of first flush passage 11 at the distal end 51 of tip 50, and also emerges from the distal end 16 of the second flush passage 12, which is located at the distal end 73 of retractable sheath 70.

The present description is for illustrative purposes only, and should not be construed to narrow the breadth of the present disclosure in any way. Thus, those skilled in the art will appreciate that various modification might be made to the presently disclosed embodiments without departing from the full and fair scope and spirit of the present disclosure. Other aspects, features and advantages will be apparent upon an examination of the attached drawings and appended claims.

What is claimed is:

1. A stent delivery system comprising:
a hub;
a cannula having an unbroken inner surface, an unbroken outer surface, a proximal end attached to the hub, and a distal carrier segment;
a tip attached to the distal carrier segment;
a pusher coaxially mounted about the cannula, and having a proximal end attached to the hub;
a retractable sheath coaxially mounted about the cannula and the pusher, and being movable between a first position covering the distal carrier segment and a second position uncovering the distal carrier segment;
a self expanding stent mounted about the distal carrier segment and covered by the sheath in the first position;
the stent delivery system includes exactly one flushing port which is defined by the hub, in fluid communication with a lumen of the cannula and an annular passage between the cannula and the pusher.

2. The stent delivery system of claim 1 including a flushing fluid connection extending between the annular passage and the distal carrier segment.

3. The stent delivery system of claim 2 wherein the flushing fluid connection includes a side port defined by the pusher.

4. The stent delivery system of claim 1 wherein the cannula is a metallic tube.

5. The stent delivery system of claim 4 wherein the cannula is a nitinol tube.

6. The stent delivery system of claim 1 wherein the hub defines at least one wire port.

7. The stent delivery system of claim 1 including a first flush passage that starts at the flushing port, extends through the lumen of the cannula, and terminates at a distal end of the tip;
a second flush passage that extends from the flushing port, along the annular passage, between the distal carrier segment and the retractable sheath, and terminates at a distal end of the retractable sheath; and
means for channeling a substantial fraction of flushing fluid through the second flush passage.

8. The stent delivery system of claim 7 wherein the means for channeling includes a removable plug in contact with the tip and at least partially blocking the first flush passage.

9. The stent delivery system of claim 1 wherein the hub defines a segment of the second flush passage that is oriented perpendicular to a centerline of the cannula.

10. The stent delivery system of claim 1 wherein the hub includes exactly two plastic bodies joined end to end.

11. A method of flushing a stent delivery system comprising the steps of:
connecting a syringe to a flushing port of the stent delivery system;
moving flushing fluid from the syringe into a first flush passage and a second flush passage defined partially by a hub that includes the flushing port;
continuing the moving step until flushing fluid emerges from a distal end of the first flush passage and a distal end of the second flush passage; and
wherein the moving step includes moving a first volume of flushing fluid along an unbroken inner surface of a cannula of the stent delivery system, and moving a second volume of flushing fluid, which is exclusive of the first volume, along an unbroken outer surface of the cannula.

12. The method of claim 11 including encouraging flushing fluid into the second fluid passage by at least partially blocking the first fluid passage.

13. The method of claim 12 wherein the blocking step includes at least partially blocking a distal end of the first flush passage with a stopper in contact with a tip of the stent delivery system.

14. The method of claim 11 wherein a distal end of the first flush passage is located at a distal end of a tip of the stent delivery system; and
the distal end of the second flush passage is located at a distal end of a retractable sheath of the stent delivery system.

15. The method of claim 11 wherein the second volume of flushing fluid moves perpendicular to a centerline of the cannula in a segment of the second flush passage defined by the hub.
